(19) United States
(12) Patent Application Publication
(10) Pub. No.: US 2018/0271657 A1
(43) Pub. Date: Sep. 27, 2018

(54) IMPLANT

(71) Applicant: PROSPER CO., LTD., Kashiwazaki-shi, Niigata (JP)

(72) Inventor: Masanori MIZUKAMI, Kashiwazaki-shi, Niigata (JP)

(21) Appl. No.: 15/763,776

(22) PCT Filed: Oct. 8, 2015
(86) PCT No.: PCT/JP2015/078638

§ 371 Date: Mar. 27, 2018

(51) Int. Cl. A61F 2/28; A61B 17/80

(52) U.S. Cl. CPC A61F 2/2875; A61B 17/8061; A61B 17/8085; A61F 2002/30115; A61F 2310/00017; A61F 2310/00407; A61F 2310/00023

(57) ABSTRACT

For the purpose of providing a flexible implant in a plane direction which prevents a scalp from sinking into an osteotomy line after surgery when joining or reconstructing the bone, implant 1 used for bone joining or reconstruction includes base units 11. The base unit 11 includes a first annular ring 10, a second annular ring 20 positioned opposite the first annular ring, and a first connecting portion 41 connecting the first annular ring 10 and the second annular ring 20. The basic units 11 are linearly linked by a second connecting portion 42 such that a first annular ring row 101 formed by a plurality of the first annular rings 10 and a second annular ring row 201 formed by a plurality of the second annular rings 20 are in parallel, and the basic units form a mesh that is capable of meandering along an osteotomy line.

### TECHNICAL FIELD

The present invention relates to an implant, in particular an implant for osteosynthesis.

### BACKGROUND ART

Surgical operations related to bones include, in addition to joining bones fractured by accident, reconstruction performed by temporarily incising bone fragment to perform surgery on organs protected by bones, and returning the bone fragment to the original position thereof. Such bone incision/reconstructive surgery is also performed as a countermeasure to lesions related to the brain with respect to the skull (see, for example, Patent Documents 1 and 2).

For example, in a surgical operation to remove a tumor of a pituitary gland, there is a method (transsphenoidal surgery) of accessing the pituitary gland via a butterfly sinus located at the back of a nose and located in front of the pituitary gland; however, a method (craniotomy) of cutting the skin and a bone of a head to access the pituitary through the gap between the brain and the brain is also frequently adopted taking into account various surgical conditions.

Incidentally, it is strongly desired not to give inconvenience or exterior distortion to patients for their normal social life after such surgery; however, there is a problem that the skin sinks into a gap of an osteotomy line generated due to a bone junction or a bone reconstruction. Especially, in a head where a scalp and a skull are in contact, there is a problem that the scalp easily sinks into the gap of the osteotomy line.

In order to solve such a situation, it is known to prepare a sheet-shaped implant, which is previously matched to the shape of the planned osteotomy line, to cover the osteotomy line. Such an implant is known which is constructed as a thin mesh and is flexible in a vertical direction seen in a side view. However, when the osteotomy line is not formed as planned in a lateral direction in a plan view, it becomes necessary to replace the implant with another implant, or to use an implant, which is greater than required and is prepared in advance, to avoid such an inconvenience.

### CITATION LIST

### Patent Literature 1

[PTL 1]
Japanese Utility Model Registration No. 3197282

[PTL 2]
Japanese Laid-open Patent Publication No. 2009-538686

### SUMMARY

### Technical Problem

In view of such circumstances, an object of the present invention is to provide a flexible implant in the planar direction in performing a bone junction or a bone reconstruction, which prevents a scalp from sinking into an osteotomy line after surgery.

### Solution to Problem

The present invention is grasped by the following constitution.

(1) According to a first aspect of the present invention is characterized in an implant for a bone junction or a bone reconstruction, comprising base units, wherein the base unit includes a first annular ring, a second annular ring positioned opposite the first annular ring, and a first connecting portion connecting the first annular ring and the second annular ring, and the basic units are linearly linked by a second connecting portion such that a first annular ring row formed by a plurality of the first annular rings and a second annular ring row formed by a plurality of the second annular rings are in parallel, and the basic units form a mesh that is capable of meandering along an osteotomy line.

(2) In (1), the first connecting portion and the second connecting portion may be formed in a straight line or curved line.

(3) In (1) or (2), the basic unit may further include a circular portion in an intermediate region of the first connecting portion, the circular portion being of a penetrating or non-penetrating type, and a circular portion row formed by a plurality of the circular portions may be parallel to the first annular ring row and the second annular ring row.

(4) In (3), the second annular ring row and the circular portion row may have an offset in a longitudinal direction with respect to the first annular ring row, the first connecting portion may include a first curved portion and a second curved portion, the first curved portion being convex to a first end side in the longitudinal direction and connecting the first annular ring and the circular portion, the second curved portion being convex to the second end side in the longitudinal direction and connecting the circular portion and the second annular ring, and the second connecting portion may include an oblique linear portion or a third curved portion, the oblique linear portion connecting the first annular ring and another circular portion adjacent to the first end side, the third curved portion being convex to the first end side.

(5) In any one of (1) through (4), the osteotomy line may be formed in a skull.

(6) In any one of (1) through (5), the basic unit may be formed of at least one of titanium, surgical grade stainless steel, steel coated with titanium, titanium nitride, a titanium alloy with another metal, and a resin containing PEEK or polyethylene.

(7) In any one of (1) through (6), the basic unit may have a thickness that is greater than or equal to 0.3 mm and less than or equal to 0.6 mm.

### Advantageous Effects of Invention

According to the present invention, it is possible to provide a flexible implant in the planar direction in performing a bone junction or a bone reconstruction, which prevents a scalp from sinking into an osteotomy line after surgery.

### BRIEF DESCRIPTION OF DRAWINGS

FIG. 1 is a diagram explaining a case where an application site of an embodiment according to the present invention is a forehead of a cranium.

FIG. 2 includes diagrams (a) to (g) illustrating examples of a basic unit employed in an implant of the embodiment according to the present invention.

FIG. 3 is a diagram illustrating an example of a linear mesh adopted in the implant of the embodiment according to the present invention.

FIG. 4 includes diagrams (a) and (b) where (a) illustrates a case where the linear mesh of FIG. 3 is arranged along an annular osteotomy line, and (b) illustrates a case where the linear mesh of FIG. 3 is arranged along an S-shaped osteotomy line.

FIG. 5 is a diagram illustrating how the implant according to the embodiment of the present invention is attached to a bone.

FIG. 6 is a diagram illustrating a modified example of the linear mesh illustrated in FIG. 3.

### DESCRIPTION OF EMBODIMENTS

Hereinafter, a mode for carrying out the present invention (hereinafter referred to as “embodiment”) will be described in detail with reference to the accompanying drawings. Note that the same reference numerals are attached to the same elements throughout the description of the embodiment.

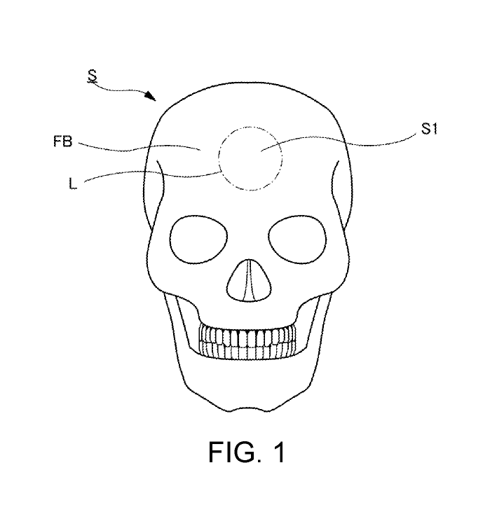

FIG. 1 is a diagram illustrating a case where an application site of an implant 1 according to the present embodiment is a frontal bone FB of a skull S, and a case is assumed, as an example, where an operation of an anterior communicable aneurysm by a trans-cerebral hemispheric fissure from the anterior side. Here, as a treated osteotomy line L, a circular line in a frontal bone FB is exemplified. The implant 1 according to the present embodiment is placed along the osteotomy line L to join or reconstruct a bone fragment S1. Of course, the implant 1 according to the present embodiment can be applied not only to the site illustrated here but also to other sites.

As will be described in detail later, the implant 1 has a linear mesh structure and can be attached in a meandering fashion along a curved osteotomy line L as illustrated in FIG. 1. The form of the curving is not limited to a specific shape, and the implant 1 can be applied not only to the illustrated circle shape but also to a C shape or the like, and the implant 1 can be suitably applied to an S shape that meanders.

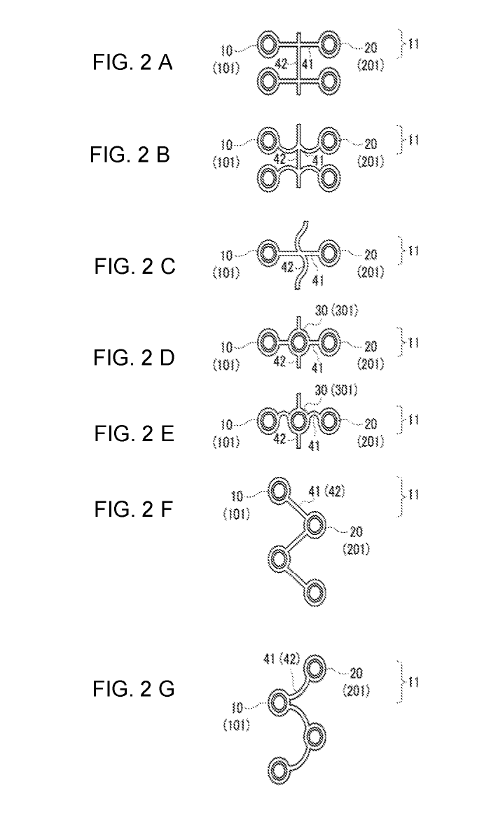

Specifically, as illustrated in FIG. 2 (a), the basic unit 11 a first annular ring 10, a second annular ring 20 positioned opposite the first annular ring 10, and a first connecting portion 41 connecting the first annular ring 10 and the second annular ring 20, and the basic units 11 are linearly linked by a second connecting portion 42 such that a first annular ring row 101 formed by a plurality of the first annular rings 10 and a second annular ring row 201 formed by a plurality of the second annular rings 20 are in parallel. In FIG. 2 (a), a case is illustrated where two basic units 11 are connected, both the first connecting portion 41 and the second connecting portion 42 are linear, and the second connecting portion 42 connects intermediate areas of the connecting portions 41. With this arrangement, by bending at least one of the right and left of the first connecting portion 41 and the second connecting portions 42 in the planar direction, it becomes possible to make the implant 1 follow the curved or meandering osteotomy line L.

FIG. 2 (b) illustrates a case where the first 1c connecting portion 41 is curved across the second connecting portion 42 in FIG. 2 (a). With this arrangement, the left and right sides of the first connecting portion 41 are curved in advance, so that the bending in the planar direction of the left and right of the first connecting portion 41 and the second connecting portion 42 in accordance with the situation of the osteotomy line L can be reduced.

FIG. 2 (c) illustrates a case where the second connecting portion 42 in FIG. 2 (a) is curved. Again, also in this case, since the second connecting portion 42 is curved in advance, it is possible to reduce the bending process of the left and right of the first connecting portion 41 and the second connecting portion 42 according to the situation of the osteotomy line L. Note that, in FIG. 2 (c), the case where there is one basic unit 11 is exemplified, but the basic units 11 are connected to the front and rear (up and down in the figure) of the illustrated basic unit 11 (in this respect, this also applies to FIG. 2 (d) and FIG. 2 (e)).

FIG. 2 (d) illustrates a case where a circular portion 30 (circular portion row 301), which is penetrated, is provided at an intermediate region of the first connecting portion 41 in FIG. 2 (a), that is, between the first annular ring 10 (the first annular ring row 101) and the second annular ring 20 (the second annular ring row 201). In the case where the annular rings are provided in three rows in this manner, even if the osteotomy line L may be meandering or zigzag with a short distance, the annular ring in any two of the three rows can be attached to the bone by passing the screw SW therethrough, and flexibility is given to the range of the treatment, which is convenient. Depending on the situation of the osteotomy line L, there is a case where the screw SW cannot be passed through the circular portion 30 provided in the intermediate region, and in order to prepare for such a case, the circular portion 30 may be non-penetrated, that is, in a disk shape, rather than penetrated, that is, annular.

FIG. 2 (e) illustrates a case where the first connecting portion 41 is curved on the left and right sides of the circular portion 30 in FIG. 2 (d). The utility is substantially the same as in the case of FIG. 2 (b). Although not illustrated in the figure, as another example, the second connecting portion 42 may be curved as in the case of FIG. 2 (c), in addition to the circular portion 30 being provided.

In FIG. 2 (f), the first annular ring 10 (the first annular ring row 101) and the second annular ring 20 (the second annular ring row 201) are arranged to take an offset position. In this case, the first connecting portion 41 connecting the first annular ring 10 and the second annular ring 20 becomes oblique straight, and as a result, the first connecting portion 41 serves as the second connecting portion 42. When the first annular ring 10 (the first annular ring row 101) and the second annular ring 20 (the second annular ring row 201) are offset in this manner, even if the osteotomy line L is bent at a steep angle, bending in the planar direction can be made small, which makes it easier for the implant 1 to follow the osteotomy line L.

FIG. 2 (g) illustrates a case where the first connecting portions 41 in FIG. 2 (f) are curved. The utility is substantially the same as in the case of FIG. 2 (b). Note that, although not illustrated, as other examples, in FIG. 2 (f) and FIG. 2 (g), a circular portion 30 may be provided in the same manner as in FIG. 2 (d) and FIG. 2 (e).

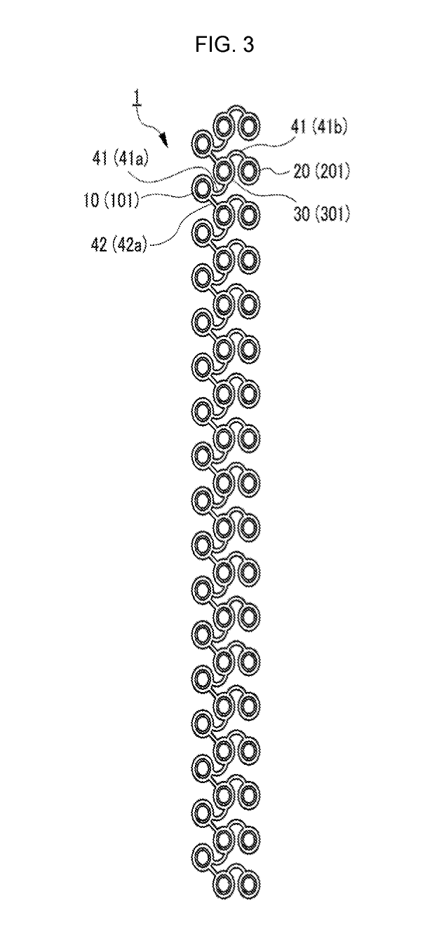

Next, with reference to FIG. 3, an embodiment, in which a combination of the first annular ring 10, the second annular ring 20, the circular portion 30, the first connecting portion 41, and the second connecting portion 42 as described above is applied, is explained.

In FIG. 3, the implant 1 is in a position in which the second annular ring row 201 and the circular portion row 301 are longitudinally offset with respect to the first annular ring row 101. Here, focusing on a second end B side, assuming a first end A side on the left side in the longitudinal direction (the lower side in the drawing) of the implant 1 and the second end B side on the right side (the upper side in the drawing), the first connecting portion 41 of the basic unit 11 includes a first curved portion 41a which is convex to the first end A in the longitudinal direction and connects the first annular ring 10 and the circular portion 30, and a second curved portion 41b which is convex to the second end B side in the longitudinal direction and connects the circular portion 30 and the second annular portion 20. The second connecting portion 42 includes an oblique linear portion 42a connecting the first annular ring 10 and the other circular portion 30 adjacent thereto on the first end A side. Here, although the circular portion 30 is penetrated, that is, in an annular shape, it may be in a non-penetrated disk shape as described above. Further, instead of the oblique linear portion 42a, the second connecting portion 42 may include a third curved portion 42b convex to the first end side A in the longitudinal direction.

The implant 1 illustrated in FIG. 3 exemplifies, as an example, a case where the number of the first annular rings 10 is 17. In this case, the number of the second annular rings 20 and the number of the circular portions 30 is 18, respectively. The width of the implant 1 (lateral length in the figure) is 8 mm, and the length (longitudinal length in the figure) is 100 mm. Of course, the number and overall dimensions of these annular rings may be arbitrarily set according to the purpose of use and the site to be applied.

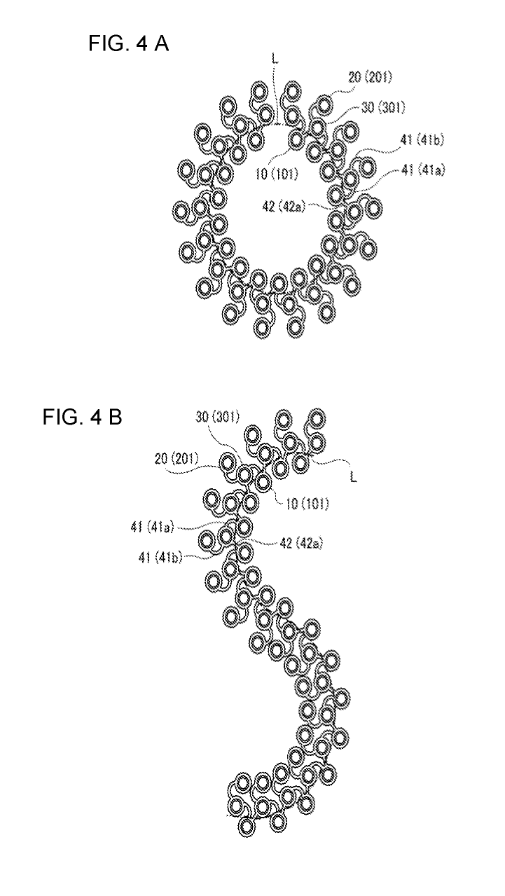

FIG. 4 illustrates a case where this implant 1 is attached along the osteotomy line L, where (a) illustrates a case of a circular shape and (b) illustrates a case of a S shape. As described above, in the implant 1, the second annular ring row 201 and the circular portion row 301 have the offset in the longitudinal direction with respect to the first annular ring row 101, the first connecting portion 41 is curved (in an S shaped when viewing the first curved portion 41a and the second curved portion 42b in a connected state), and the second connecting portion 42 is formed to have an oblique linear shape, which makes it very easy for the implant 1 to follow the curved or meandering osteotomy line L.

Here, the materials constituting the implant 1 according to the present embodiment is described. It is preferable that the implant 1 is made of titanium which is highly compatible with living bodies and is minimally invasive. However, even if it is not necessarily pure titanium, any one of surgical grade stainless steel, titanium coated steel, titanium nitride, other metals (for example, a titanium-nickel alloy or the like), and a resin containing PEEK or polyethylene may be used to adjust physical characteristics of a metal according to the necessity of particular purposes. In any event, it suffices as long as the implant 1 can be at least partly bent while maintaining its structural integrity.

The thickness of the implant 1 is preferably from 0.3 mm to 0.6 mm, more preferably from 0.3 mm to 0.4 mm, most preferably 0.3 mm, in order to suppress irritation to the scalp with low invasiveness. The implant 1 according to the present embodiment further achieves a smooth surface without corners by performing edge polishing over the entire periphery, and suppresses irritation to the scalp to the utmost.

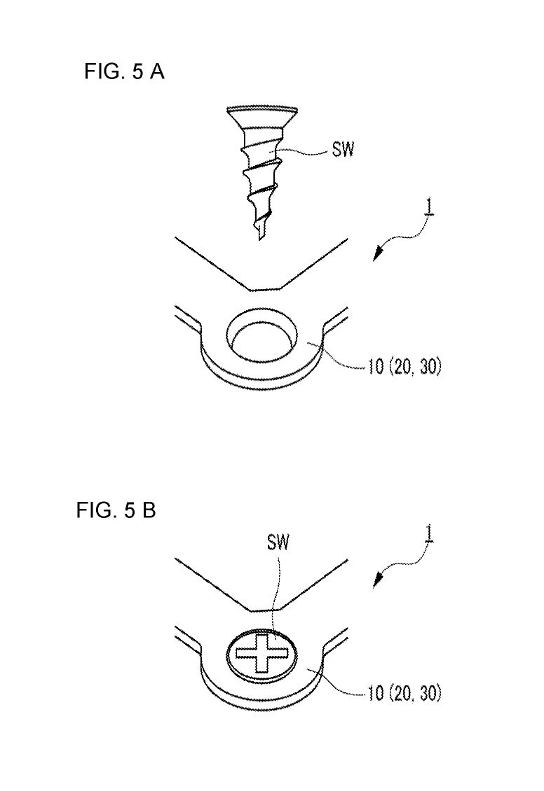

Further, as illustrated in FIG. 5, with respect to the screw SW to be passed through in the annular ring, the top surface of the head is flattened so that the protrusion from the implant 1 is suppressed so that it cannot be substantially tactile. Note that the shape of the screw SW is configured by taking into account the first bite into the bone, the break of the screw edge, and the prevention of the last spinning around.

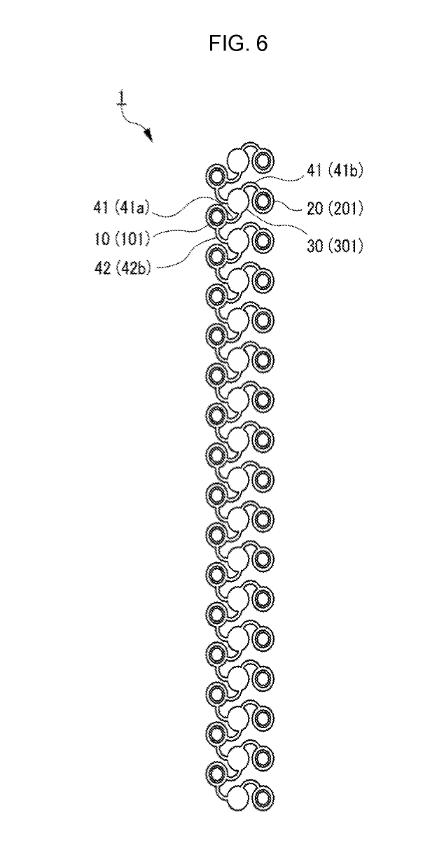

As described above, the implant 1 according to the present embodiment may have a circular portion 30 of a non-penetrated disk shape and a second connection portion 42 as a third curved portion 42b protruding toward the first end A side in the longitudinal direction. Such a configuration is illustrated in FIG. 6 as a modified example.

Although the present invention has been described with reference to the embodiments, it is needless to say that the technical scope of the present invention is not limited to the scope described in the above embodiment. It is obvious to a person skilled in the art that various modifications or improvements can be added to the above embodiment. It is obvious from the description of the scope of claims that configurations with such modification or improvement can be included in the technical scope of the present invention.

For example, the application of the implant 1 according to the present embodiment is not limited to the application of the frontal bone FB of the skull S assuming surgery for an anterior communicating aneurysm by a trans-cerebral hemispheric fissure approach from the anterior side, it can be applied to other parts of the skull and bones other than the skull.

### Description of Reference Symbols

1 implant
10 first annular ring
101 first annular ring row
20 second annular ring
201 second annular ring row
30 circular portion
301 circular portion low
41 first connecting portion
41a first curved portion
42 second connecting portion
42b second curved portion
S skull
FB frontal bone
L osteotomy line

1. An implant for a bone junction or a bone reconstruction, comprising base units, wherein the base unit includes
a first annular ring,
a second annular ring positioned opposite the first annular ring, and
a first connecting portion connecting the first annular ring and the second annular ring, and
the basic units are linearly linked by a second connecting portion such that a first annular ring row formed by a plurality of the first annular rings and a second annular ring row formed by a plurality of the second annular rings are in parallel, and the basic units form a mesh that is capable of meandering along an osteotomy line,
the basic unit further includes a circular portion in an intermediate region of the first connecting portion, the circular portion being of a penetrating or non-penetrating type, and
a circular portion row formed by a plurality of the circular portions is parallel to the first annular ring row and the second annular ring row,
the second annular ring row and the circular portion row have an offset in a longitudinal direction with respect to the first annular ring row,
the first connecting portion includes a first curved portion and a second curved portion, the first curved portion being convex to a first end side in the longitudinal direction and connecting the first annular ring and the circular portion, the second curved portion being convex to the second end side in the longitudinal direction and connecting the circular portion and the second annular ring, and
the second connecting portion includes an oblique linear portion or a third curved portion, the oblique linear portion connecting the first annular ring and another circular portion adjacent to the first end side, the third curved portion being convex to the first end side.

2. The implant according to claim 1, wherein the first connecting portion and the second connecting portion are formed in a straight line or curved line.

3-4. (canceled)

5. The implant according to claim 1, wherein the osteotomy line is formed in a skull.

6-7. (canceled)

8. The implant according to claim 2, wherein the osteotomy line is formed in a skull.

9. The implant according to claim 1, wherein the basic unit is formed of at least one of titanium, surgical grade stainless steel, steel coated with titanium, titanium nitride, a titanium alloy with another metal, and a resin containing PEEK or polyethylene.

10. The implant according to claim 2, wherein the basic unit is formed of at least one of titanium, surgical grade stainless steel, steel coated with titanium, titanium nitride, a titanium alloy with another metal, and a resin containing PEEK or polyethylene.

11. The implant according to claim 5, wherein the basic unit s formed of at least one of titanium, surgical grade stainless steel, steel coated with titanium, titanium nitride, a titanium alloy with another metal, and a resin containing PEEK or polyethylene.

12. The implant according to claim 8, wherein the basic unit is formed of at least one of titanium, surgical grade stainless steel, steel coated with titanium, titanium nitride, a titanium alloy with another metal, and a resin containing PEEK or polyethylene.

13. The implant according to claim 1, wherein the basic unit has a thickness that is greater than or equal to 0.3 mm and less than or equal to 0.6 mm.

14. The implant according to claim 2, wherein the basic unit has a thickness that is greater than or equal to 0.3 mm and less than or equal to 0.6 mm.

15. The implant according to claim 5, wherein the basic unit has a thickness that is greater than or equal to 0.3 mm and less than or equal to 0.6 mm.

16. The implant according to claim 8, wherein the basic unit has a thickness that is greater than or equal to 0.3 mm and less than or equal to 0.6 mm.

17. The implant according to claim 9, wherein the basic unit has a thickness that is greater than or equal to 0.3 mm and less than or equal to 0.6 mm.

18. The implant according to claim 10, wherein the basic unit has a thickness that is greater than or equal to 0.3 mm and less than or equal to 0.6 mm.

19. The implant according to claim 11, wherein the basic unit has a thickness that is greater than or equal to 0.3 mm and less than or equal to 0.6 mm.

20. The implant according to claim 12, wherein the basic unit has a thickness that is greater than or equal to 0.3 mm and less than or equal to 0.6 mm.
